(19) United States
(12) Patent Application Publication
(10) Pub. No.: US 2014/0060533 A1
(43) Pub. Date: Mar. 6, 2014

(54) METHODS AND SYSTEM FOR HUMIDIFIER CONTROL USING ENTHALPY-BASED CALCULATION

(71) Applicant: FISHER & PAYKEL HEALTHCARE LIMITED, Auckland (NZ)

(72) Inventors: Guohua Bao, Auckland (NZ); Yi-cheng Sun, Auckland (NZ)

(73) Assignee: FISHER & PAYKEL HEALTHCARE LIMITED, Auckland (NZ)

(21) Appl. No.: 14/014,130

(22) Filed: Aug. 29, 2013

Related U.S. Application Data
(60) Provisional application No. 61/695,725, filed on Aug. 31, 2012.

(51) Int. Cl. A61M 16/16; A61M 16/10; A61M 16/00

(52) U.S. Cl. CPC A61M 16/16; A61M 16/0057; A61M 16/1075
USPC 128/203.14

(57) ABSTRACT

A respiratory humidification system that controls the humidity of the outlet gases using an enthalpy based calculation is disclosed herein. The respiratory humidification system includes a controller that can receive data from one or more of an enthalpy sensor, a flow sensor and a temperature sensor. The controller can also receive information relating to the outlet gas enthalpy, which can be calculated from the desired output temperature and humidity. A level of power to be provided to the heating system to achieve the desired output temperature and humidity can be calculated using data from the enthalpy sensor, flow sensor, and temperature sensor, as well as the calculated outlet gas enthalpy.

### BACKGROUND OF THE INVENTION

1. Field of the Invention

The present invention relates generally to respiratory therapy. In particular, the present invention relates to respiratory humidifier control.

2. Description of the Related Art

Devices or systems for providing a humidified gases flow to a patient for therapeutic purposes are known in the art. Gases are delivered to a humidification chamber from a gases source and as the gases pass over hot water, or through the heated, humidified air in the humidification chamber, the gases become saturated with water vapour. The heated humidified gases are then delivered to a user or patient downstream from the humidification chamber, via a gases conduit and a user interface.

The gases delivery system can include a gases source, such as an assisted breathing unit (e.g., ventilator) or a blower unit. The gases source and the humidification chamber can be separate, modular units that can be connected in series via connection conduits to allow gases to pass from the gases source to the humidifier unit. Alternatively, the gases delivery system can be an integrated system, where the gases source and the humidification chamber are contained within the same unit or housing in use. In both modular and integrated systems, the gases provided by the gases source are generally sourced from the surrounding atmosphere.

Another type of gases delivery system, which is typically used in hospitals, is one where the gases delivery system receives at least a portion of the gases which it uses from a central gases source. A gases conduit or similar is connected between an inlet which is typically mounted in the wall of a patients room. The gases conduit is either connected directly to the humidification chamber in use, or a pressure regulator unit or similar can be connected in series between the gases inlet and the humidification chamber if required.

It is desirable to control the characteristics of the gases delivered to the patient at the user interface to provide the required therapy. Some of the characteristics of the gases that are controlled include temperature, humidity, flow, and oxygen fraction (if the patient is undergoing oxygen therapy). A therapy regime can become ineffective if the gases are not delivered to the patient with the correct or required characteristics. Often, the gases that are delivered are fully saturated with water vapour (i.e. at substantially 100% relative humidity) to a user, at a constant flow rate. Other types or variations of therapy regime may call for less than 100% relative humidity.

Breathing circuits are not steady-state systems, and it is difficult to ensure the gases are delivered to a user with substantially the correct characteristics. It can be difficult to achieve this result over a range of ambient temperatures, ambient humidity levels, and a range of gas flows at the point of delivery. The temperature, flow rate and humidity of a gases stream are all interdependent characteristics. When one characteristic changes, the others may also change. A number of external variables can affect the gases within a breathing circuit and make it difficult to deliver the gases to the user at substantially the right temperature, flow rate and humidity. As one example, the delivery conduit between the patient and the humidifier outlet is exposed to ambient atmospheric conditions, and cooling of the heated, humidified gases within the conduit can occur as the gas travels along the conduit between the exit port of the humidification chamber and the user interface. This cooling can lead to ‘rain-out’ within the conduit, which is condensate forming on the inner surface of the conduit.

In order to assist in achieving delivery of gases having the desired characteristics, prior systems have used sensors (e.g., temperature and humidity sensors) located at various positions throughout the breathing circuit. Thermistors are generally used as temperature sensors, as these are reliable and inexpensive. Humidity sensors can be suitable for use with systems that deliver heated humidified gases to a patient for therapeutic purposes.

Patent publication WO2001/13981 describes a system for using the output of these sensors to control aspects of the humidified gases supply system. Patent publication WO 2009/145646 is another system for using the output of sensors to control aspects of the humidified gases supply system. The contents of these publications are hereby incorporated by reference herein, in their entireties.

### SUMMARY OF THE DISCLOSURE

An aspect of at least one of the embodiments disclosed herein includes the realization that the output of a humidified gases supply system can be controlled by knowing the enthalpy of the gases at the inlet of the system and calculating the desired enthalpy of the gases to be delivered to the patient.

Thus, in accordance with at least one of the embodiments disclosed herein, a respiratory humidification system with an inlet and an output can have a flow source in fluid communication with the inlet. The respiratory humidification system can include a humidifier having a humidification chamber and a heating system, and the humidifier can be in fluid communication with the flow source. The respiratory humidification system can further include a controller, an enthalpy sensor configured to measure an inlet gas enthalpy; and a flow sensor configured to measure a gas flow rate.

The enthalpy sensor can be disposed at or near the inlet of the respiratory humidification system. In some embodiments, the enthalpy sensor can be disposed at or near the inlet of the humidifier.

The respiratory humidification system can further include at least one temperature sensor configured to measure a gas temperature. The at least one of the temperature sensors can be disposed at or near the inlet of the respiratory humidification system.

The controller can be configured to calculate a power to be applied to the humidifier using at least one or more of an output gas enthalpy, the inlet gas enthalpy, and the gas flow rate.

The output gas enthalpy can be calculated at least partially from a desired output temperature and a desired output humidity of the respiratory humidification system. The power to be applied to the humidifier can be calculated using the equation

$${P}_{input} = \frac{( {h}_{output} - {h}_{inlet} ) \cdot F}{η}$$

where Pᵢₙₚᵤₜ is the power to be applied, hₒᵤₜₚᵤₜ is the output gas enthalpy, hᵢₙₗₑₜ is the inlet gas enthalpy, F is the gas flow rate and η is a power efficiency of the heating system.

In some embodiments, the heating system can include system.

In some embodiments, the respiratory humidification system can be a continuous positive airway pressure (CPAP) apparatus.

In accordance with at least one of the embodiments disclosed herein, a method of controlling a heating system of a humidifier in a respiratory humidification system can include: obtaining one or more of a first enthalpy data from an enthalpy sensor and flow rate data from a flow sensor; obtaining a second enthalpy data that is calculated at least partially from a desired output temperature and a desired output humidity of the respiratory humidification system; calculating a power level of the heating system corresponding to the desired output temperature and/or desired output humidity based on at least one or more of the first enthalpy data, the second enthalpy data and the flow rate data; and supplying the calculated power level to the heating system.

The first enthalpy data can be measured at or near the inlet of the respiratory humidification system. In some embodiments, the first enthalpy data can be measured at or near the inlet of the humidifier.

In some embodiments, the method of controlling the heating system can include obtaining temperature data from a temperature sensor. The temperature data can be measured at or near the inlet of the respiratory humidification system.

In some embodiments, the respiratory humidification system can be a continuous positive airway pressure (CPAP) apparatus.

In accordance with at least one of the embodiments disclosed herein, a respiratory humidification system with an inlet and an output can have a flow source in fluid communication with the inlet. The respiratory humidification system can include a humidifier having a humidification chamber and a heating system, the humidifier in fluid communication with the flow source. The respiratory humidification system can further include a controller, a capacity sensor configured to measure a gas dielectric constant, a flow sensor configured to measure a gas flow rate, and at least one temperature sensor configured to measure a gas temperature.

The capacity sensor can be disposed at or near the inlet of the respiratory humidification system. In some embodiments, the capacity sensor can be disposed at or near the inlet of the humidifier. In some embodiments, at least one of the temperature sensors can be disposed at or near the inlet of the respiratory humidification system.

The controller can be configured to calculate the enthalpy of gases entering the inlet of the respiratory humidification system using data obtained from the capacity sensor and the at least one temperature sensor. The calculated enthalpy can be used along with data obtained from the flow sensor to control the amount of power provided to the heating system of the humidifier.

The controller can be configured to calculate a power to be applied to the humidifier using at least one or more of an output gas enthalpy, the gas dielectric constant, the gas flow rate and the gas temperature. The output gas enthalpy can be calculated at least partially from a desired output temperature and a desired output humidity of the respiratory humidification system.

The power to be applied to the humidifier can be calculated using the equation

$${P}_{input} = \frac{( {h}_{output} - {h}_{inlet} ) \cdot F}{η}$$

where Pᵢₙₚᵤₜ is the power to be applied, hₒᵤₜₚᵤₜ is the output gas enthalpy, hᵢₙₗₑₜ is an inlet gas enthalpy calculated using the gas dielectric constant and the gas temperature, F is the gas flow rate and η is a power efficiency of the heating system.

In some embodiments, the heating system can include a heater plate.

In some embodiments, the respiratory humidification system can be a continuous positive airway pressure (CPAP) apparatus.

In accordance with at least one of the embodiments disclosed herein, a method of controlling a heating system of a humidifier in a respiratory humidification system can include: obtaining one or more of a capacitance data from a capacity sensor, temperature data from a temperature sensor, and flow rate data from a flow sensor; obtaining an output gas enthalpy data that is calculated at least partially from a desired output temperature and a desired output humidity of the respiratory humidification system; calculating a power level of the heating system corresponding to the desired output temperature and/or desired output humidity based on at least one or more of the capacitance data, the output gas enthalpy data, the temperature data and the flow rate data; and supplying the calculated power level to the heating system.

The capacitance data can be measured at or near the inlet of the respiratory humidification system. In some embodiments, the capacitance data can be measured at or near the inlet of the humidifier.

The temperature data can be measured at or near the inlet of the respiratory humidification system.

In some embodiments, the respiratory humidification system can be a continuous positive airway pressure (CPAP) apparatus.

### BRIEF DESCRIPTION OF THE DRAWINGS

Specific embodiments and modifications thereof will become apparent to those skilled in the art from the detailed description herein having reference to the figures that follow, of which:

FIG. 1 is an overview diagram of a control arrangement for a humidification system or an apparatus including a humidifier, such as a continuous positive airway pressure (CPAP) apparatus.

FIG. 2 is a humidification system diagram in which enthalpy of the breathing gas is measured, such as at a location at or near the inlet.

FIG. 3 is a humidification system diagram in which enthalpy of the breathing gas is calculated by the measurement of another property of the breathing gas, such as the dielectric constant, which can be measured, for example, at a location at or near the inlet.

### DETAILED DESCRIPTION OF SPECIFIC EMBODIMENTS

In controlling the output humidity (e.g., relative humidity or “RH”) of a humidification system or device incorporating a humidifier, such as a continuous positive airway pressure (CPAP) device, it is possible to measure the inlet humidity or RH. However, certain issues can arise with the measurement of humidity of the gases entering the inlet including, for example, placement of the RH sensor. In particular, if a RH sensor is positioned between the blower and the chamber, it can result in some undesirable issues, such as condensation, wrong RH measurement and slow real time RH measurement.

However, gases have many properties, like temperature, absolute humidity (AH), relative humidity (RH), dew point, vapor pressure, mixing ratio, density and enthalpy. These properties have a theoretical relationship for a given pressure. For example, at least one of these properties can be calculated by knowing two of the other properties.

Enthalpy is traditionally defined as a measure of the total energy of a thermodynamic system. In regards to this disclosure, enthalpy can refer to a measure of the total energy in a gas. Enthalpy of a gas can be defined as the sum of sensible and latent heat for each component in the gas. Because the absolute enthalpy of a gas is difficult to determine, values of enthalpy are conventionally expressed relative to a datum point (for a dry gas, this is normally the heat content at 0° C.).

Enthalpy and humidity are two different concepts. Enthalpy is related to the capacity of holding energy by a gas, while humidity is related to the ability of holding of water vapor by a gas.

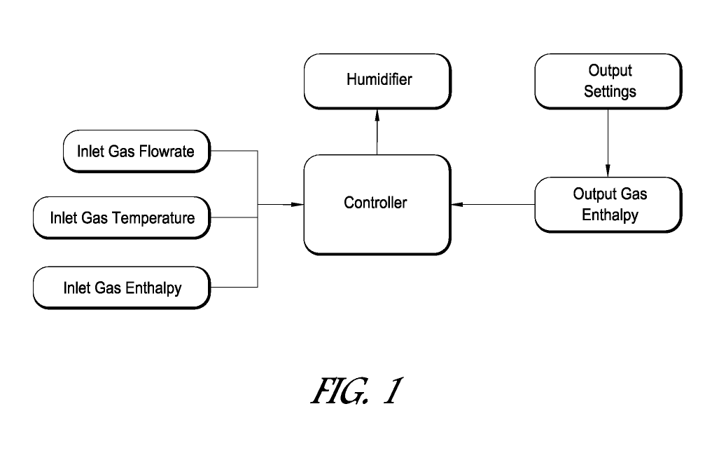

With reference to FIG. 1, the inlet gas or ambient gas enthalpy hᵢₙₗₑₜ, inlet gas or ambient gas temperature, and gas flow rate F can be measured. One or more of these measurements can be sent to a controller that controls the power to the respiratory humidification system. The desired gas output settings, such as output temperature and output humidity, are known from the comfort settings of the humidifier which can be adjusted by the user independently or in combination. The output gas enthalpy hₒᵤₜₚᵤₜ can be calculated using the output settings and the calculated output gas enthalpy can be sent to the controller. The power efficiency η of the heating system, which may include for example a heater plate, can be determined experimentally. The power Pᵢₙₚᵤₜ (e.g., heater plate power) that may be applied to the humidifier can be calculated using the following equation:

$${P}_{input} = \frac{( {h}_{output} - {h}_{inlet} ) \cdot F}{η}$$

where Pᵢₙₚᵤₜ is the power to be applied (which can be expressed in Joules/sec), hₒᵤₜₚᵤₜ is the output gas enthalpy (which can be expressed in Joules/gram), hᵢₙₗₑₜ is the inlet gas or ambient gas enthalpy (which can be expressed in Joules/gram), F is the gas flow rate (which can be expressed in grams/second, or alternatively liters/second multiplied by a conversion factor for grams/liter), and η is a power efficiency of the heating system (which has no units). From the above equation, the power Pᵢₙₚᵤₜ can be controlled to reach a desired output of humidity and temperature.

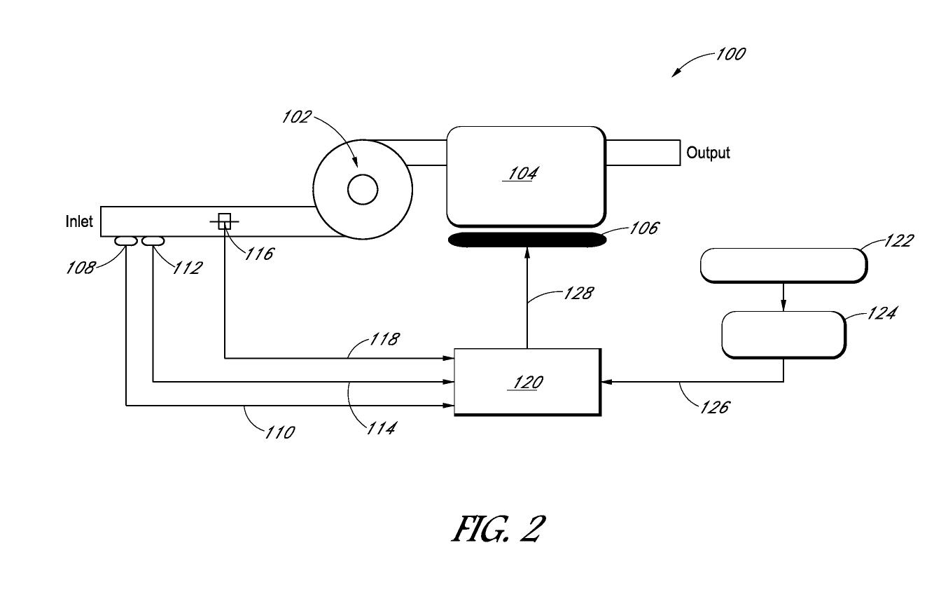

There are several ways to obtain the inlet gas enthalpy. FIG. 2 illustrates an example of a respiratory humidification system 100 that measures inlet gas enthalpy directly from an enthalpy sensor 108. The respiratory humidification system 100 includes a flow source 102, such as a blower, that is in fluid communication with the system inlet. The blower outlet is in fluid communication with an inlet of a humidifier having a humidification chamber 104 that can hold fluids for humidifying the inlet gases. The humidification chamber 104 can be in thermal communication with a heating system 106, such as a heater plate. The heating system 106 can heat and humidify the contents of the humidification chamber 104. The heated and humidified gases then exit the outlet of the humidification chamber 104 to the output, which in some instances can be the patient interface.

The power 128 to the heating system 106 is controlled by a controller 120. The controller 120 can receive enthalpy data 110 from an enthalpy sensor 108. In some embodiments, the controller 120 can receive temperature data 114 from a temperature sensor 112. The enthalpy sensor 108 and temperature sensor 112 are shown in a location at or near the inlet of the respiratory humidification system 100, however other suitable locations can be used. In some embodiments, the enthalpy sensor 108 and/or temperature sensor 112 can be disposed at or near the inlet of the humidification chamber 104, such as for example between the flow source 102 and humidification chamber 104. Locating the sensor or sensors between the flow source 102 and humidification chamber 104 can beneficially provide more accurate readings of the gases entering the humidification chamber by measuring the gases after the flow source, especially when the flow source has affected the gas temperature and the enthalpy. The controller 120 can receive flow rate data 118 from a flow sensor 116, which can be located in the flow path of the inlet gases, such as at or near the inlet of the flow source, or at or near the inlet of the humidification chamber. The user can set the output settings 122, which can include the desired humidity and temperature to be delivered to the patient. An output gas enthalpy 124 can be calculated using the output settings 122 and known equations. The output gas enthalpy data 126 can be sent to the controller 120. The controller 120 can then calculate the power 128 to be provided to the heating system 106 to achieve the desired output gas properties delivered to the patient.

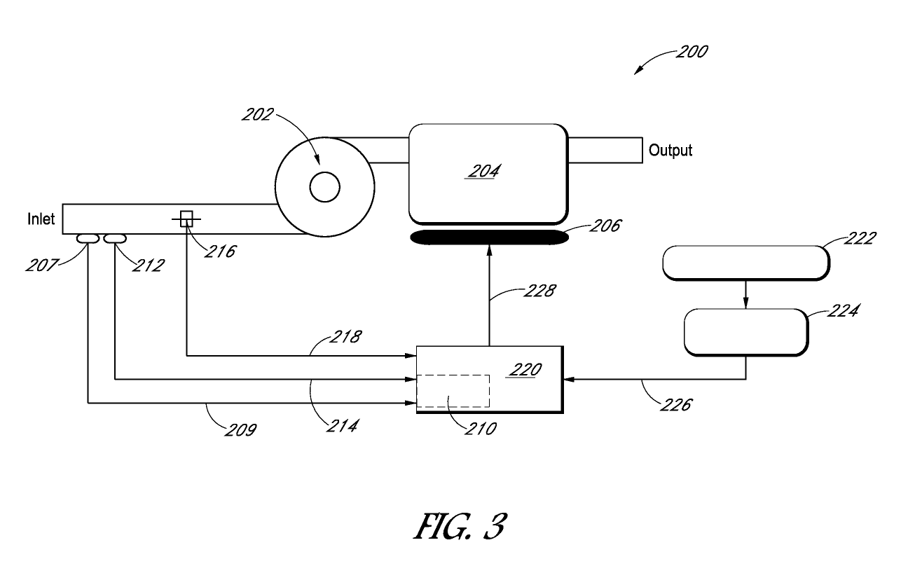

In some embodiments, the inlet gas enthalpy can be obtained by measuring a parameter of the gas that can be related to enthalpy, for example a capacity sensor that measures capacitance data such as gas dielectric constant. FIG. 3 illustrates an example of a respiratory humidification system 200 that measures gas capacitance using a capacity sensor 207 to obtain inlet gas enthalpy. The respiratory humidification system 200 includes a flow source 202, such as a blower, that is in fluid communication with the system inlet. The blower outlet is in fluid communication with an inlet of a humidifier having a humidification chamber 204 that can hold fluids for humidifying the inlet gases. The humidification chamber 204 can be in thermal communication with a heating system 206, such as a heater plate. The heating system 206 can heat and humidify the contents of the humidification chamber 204. The heated and humidified gases then exit the outlet of the humidification chamber 204 to the output, which in some instances can be the patient interface.

The power 228 to the heating system 206 is controlled by a controller 220. The controller 220 can receive capacitance data 209 from a capacity sensor 207 and temperature data 214 from a temperature sensor 212. The controller 220 can calculate the inlet gas enthalpy 210 using the capacitance data 209 and temperature data 214, because there is a relationship between gas enthalpy and measured dielectric property. The capacity sensor 207 and temperature sensor 212 are shown in a location at or near the inlet of the respiratory humidification system 200, however other suitable locations can be used. In some embodiments, the capacity sensor 207 and/or temperature sensor 212 can be disposed at or near the inlet of the humidification chamber 204, such as for example between the flow source 202 and humidification chamber 204. Locating the sensor or sensors between the flow source 202 and humidification chamber 204 can beneficially provide more accurate readings of the gases entering the humidification chamber by measuring the gases after the flow source, especially when the flow source has affected the gas temperature and capacitance. The controller 220 can receive flow rate data 218 from a flow sensor 216, which can be located in the flow path of the inlet gases, such as at or near the inlet of the flow source, or at or near the inlet of the humidification chamber. The user can set the output settings 222, which can include the desired humidity and temperature to be delivered to the patient. An output gas enthalpy 224 can be calculated using the output settings 222 and known equations. The output gas enthalpy data 226 can be sent to the controller 220. The controller 220 can then calculate the power 228 to be provided to the heating system 206 to achieve the desired output gas properties delivered to the patient.

For the above-described humidity algorithms, the focus was on the enthalpy and power control. Data processing can be related to the power/energy parameters. The method and control systems disclosed herein can be applied to any humidification device, such as any suitable respiratory humidification system or a device incorporating a humidifier, such as a CPAP device (e.g., the ICON line of CPAP devices sold by Fisher & Paykel Healthcare).

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Where, in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the scope of the invention.

Additionally, it will be recognized that the methods described herein may be practiced in different sequences, and/or with additional devices as desired. Such alternative embodiments and/or uses of the methods and devices described above and obvious modifications and equivalents thereof are intended to be included within the scope of the present invention. Thus, it is intended that the scope of the present invention should not be limited by the particular embodiments described above, but should be determined only by a fair reading of the claims that follow.

What is claimed is:

1. A respiratory humidification system with an inlet and an output, the respiratory humidification system comprising:
a flow source in fluid communication with the inlet;
a humidifier comprising a humidification chamber and a heating system, the humidifier in fluid communication with the flow source;
a controller;
an enthalpy sensor configured to measure an inlet gas enthalpy; and
a flow sensor configured to measure a gas flow rate.

2. The respiratory humidification system of claim 1, wherein the enthalpy sensor is disposed at or near the inlet of the respiratory humidification system.

3. The respiratory humidification system of claim 1, wherein the enthalpy sensor is disposed at or near the inlet of the humidifier.

4. The respiratory humidification system of claim 1, further comprising at least one temperature sensor configured to measure a gas temperature.

5. The respiratory humidification system of claim 4, wherein at least one of the temperature sensors is disposed at or near the inlet of the respiratory humidification system.

6. The respiratory humidification system of claim 1, wherein the controller is configured to calculate a power to be applied to the humidifier using at least one or more of an output gas enthalpy, the inlet gas enthalpy, and the gas flow rate.

7. The respiratory humidification system of claim 6, wherein the output gas enthalpy is calculated at least partially from a desired output temperature and a desired output humidity of the respiratory humidification system.

8. The respiratory humidification system of claim 7, wherein the power to be applied to the humidifier is calculated using the equation
$${P}_{input} = \frac{( {h}_{output} - {h}_{inlet} ) \cdot F}{η}$$
where Pᵢₙₚᵤₜ is the power to be applied, hₒᵤₜₚᵤₜ is the output gas enthalpy, hᵢₙₗₑₜ is the inlet gas enthalpy, F is the gas flow rate and η is a power efficiency of the heating system.

9. The respiratory humidification system of claim 1, wherein the heating system comprises a heater plate.

10. The respiratory humidification system of claim 1, wherein the respiratory humidification system is a continuous positive airway pressure apparatus.

11. A method of controlling a heating system of a humidifier in a respiratory humidification system, the method comprising:
obtaining one or more of a first enthalpy data from an enthalpy sensor and flow rate data from a flow sensor;
obtaining a second enthalpy data that is calculated at least partially from a desired output temperature and a desired output humidity of the respiratory humidification system;
calculating a power level of the heating system corresponding to the desired output temperature and/or desired output humidity based on at least one or more of the first enthalpy data, the second enthalpy data and the flow rate data; and
supplying the calculated power level to the heating system.

12. The method of controlling a heating system of claim 11, wherein the first enthalpy data is measured at or near the inlet of the respiratory humidification system.

13. The method of controlling a heating system of claim 11, wherein the first enthalpy data is measured at or near the inlet of the humidifier.

14. The method of controlling a heating system of claim 11, further comprising obtaining temperature data from a temperature sensor.

15. The method of controlling a heating system of claim 14, wherein the temperature data is measured at or near the inlet of the respiratory humidification system.

16. The method of controlling a heating system of claim 11, wherein the respiratory humidification system is a continuous positive airway pressure apparatus.

17. A respiratory humidification system with an inlet and an output, the respiratory humidification system comprising:
a flow source in fluid communication with the inlet;
a humidifier comprising a humidification chamber and a heating system, the humidifier in fluid communication with the flow source;
a controller;
a capacity sensor configured to measure a gas dielectric constant;
a flow sensor configured to measure a gas flow rate; and
at least one temperature sensor configured to measure a gas temperature.

18. The respiratory humidification system of claim 17, wherein the capacity sensor is disposed at or near the inlet of the respiratory humidification system.

19. The respiratory humidification system of claim 17, wherein the capacity sensor is disposed at or near the inlet of the humidifier.

20. The respiratory humidification system of claim 17, wherein at least one of the temperature sensors is disposed at or near the inlet of the respiratory humidification system.

21. The respiratory humidification system of claim 17, wherein the controller is configured to calculate the enthalpy of gases entering the inlet of the respiratory humidification system using data obtained from the capacity sensor and the at least one temperature sensor.

22. The respiratory humidification system of claim 21, wherein the calculated enthalpy is used along with data obtained from the flow sensor to control the amount of power provided to the heating system of the humidifier.

23. The respiratory humidification system of claim 17, wherein the controller is configured to calculate a power to be applied to the humidifier using at least one or more of an output gas enthalpy, the gas dielectric constant, the gas flow rate and the gas temperature.

24. The respiratory humidification system of claim 23, wherein the output gas enthalpy is calculated at least partially from a desired output temperature and a desired output humidity of the respiratory humidification system.

25. The respiratory humidification system of claim 24, wherein the power to be applied to the humidifier is calculated using the equation
$${P}_{input} = \frac{( {h}_{output} - {h}_{inlet} ) \cdot F}{η}$$
where Pᵢₙₚᵤₜ is the power to be applied, hₒᵤₜₚᵤₜ is the output gas enthalpy, hᵢₙₗₑₜ is an inlet gas enthalpy calculated using the gas dielectric constant and the gas temperature, F is the gas flow rate and η is a power efficiency of the heating system.

26. The respiratory humidification system of claim 17, wherein the heating system comprises a heater plate.

27. The respiratory humidification system of claim 17, wherein the respiratory humidification system is a continuous positive airway pressure apparatus.

28. A method of controlling a heating system of a humidifier in a respiratory humidification system, the method comprising:
obtaining one or more of a capacitance data from a capacity sensor, temperature data from a temperature sensor, and flow rate data from a flow sensor;
obtaining an output gas enthalpy data that is calculated at least partially from a desired output temperature and a desired output humidity of the respiratory humidification system;
calculating a power level of the heating system corresponding to the desired output temperature and/or desired output humidity based on at least one or more of the capacitance data, the output gas enthalpy data, the temperature data and the flow rate data; and
supplying the calculated power level to the heating system.

29. The method of controlling a heating system of claim 28, wherein the capacitance data is measured at or near the inlet of the respiratory humidification system.

30. The method of controlling a heating system of claim 28, wherein the capacitance data is measured at or near the inlet of the humidifier.

31. The method of controlling a heating system of claim 28, wherein the temperature data is measured at or near the inlet of the respiratory humidification system.

32. The method of controlling a heating system of claim 28, wherein the respiratory humidification system is a continuous positive airway pressure apparatus.
